(19) United States
(12) Patent Application Publication
(10) Pub. No.: US 2023/0233299 A1
(43) Pub. Date: Jul. 27, 2023

(54) DENTAL, ENDODONTIC, AND PERIODONTIC TREATMENT METHODS AND SYSTEMS

(71) Applicant: EdgeEndo, LLC, Albuquerque, NM (US)

(72) Inventor: Charles J. Goodis, Winter Park, FL (US)

(21) Appl. No.: 17/586,402

(22) Filed: Jan. 27, 2022

(51) Int. Cl. A61C 17/20; A61C 5/40; A61C 19/06; A61C 5/50; A61C 1/07

(52) U.S. Cl. CPC A61C 17/20; A61C 5/40; A61C 19/063; A61C 5/50; A61C 1/07

(57) ABSTRACT

In a method and system for performing a treatment on a tooth, ultrasonic energy is generated and transmitted by an ultrasonic instrument to a fluid located in the interior of the tooth. The amplitude and frequency of the ultrasonic energy causes inertial cavitation of gas vesicles located in bacteria present in the fluid to assist in cleaning, disinfecting, and/or delivering a payload to the fluid and interior of the tooth but is below the safety limit for ultrasonic imaging.

### FIELD OF THE INVENTION

The present invention relates to dental, endodontic, and periodontic treatment methods and systems.

### BACKGROUND INFORMATION

Dentistry is the branch of medicine related to the study, diagnosis, prevention, and treatment of diseases, disorders, and conditions of the oral cavity, e.g., the mouth, commonly in the dentition, e.g., the development and arrangement of teeth, and the oral mucosa, and of adjacent and related structures and tissues, particularly in associated maxillofacial, e.g., jaw and facial, area. The field of dentistry or dental medicine includes teeth as well as other aspects of the craniofacial complex including the temporomandibular joint and other supporting, muscular, lymphatic, nervous, vascular, and anatomical structures. Within the general filed of dentistry are a number of specialties, including, for example, endodontics, also referred to as endodontology, and periodontics, also referred to as periodontology.

Endodontics is the dental specialty that is concerned with the study and treatment of dental pulp, e.g., the part of the center of a tooth that includes living connective tissue and odontoblasts. Endodontic treatments focus on the removal of pulpal tissue, filing and shaping root canals, obturation of the root canal space, and permanent replacement of teeth.

Periodontics is the dental specialty that studies supporting structures of teeth and diseases and conditions that affect them. The supporting tissues are known as the periodontium, which includes the gingiva, e.g., gums, alveolar bone, cementum, and the periodontal ligament. A periodontist is a dentist that specializes in the prevention, diagnosis, and treatment of periodontal disease and in the placement of dental implants.

A number of techniques may be utilized to clean the interior portion of a tooth as part of dental, endodontic, and/or periodontic treatment regimens. For example, mechanical files and other instruments may be used for bulk removal of dental pulp, tissue, and other debris from the interior of a tooth, e.g., its root(s). However, such mechanical removal may be incomplete, in that some dental pulp, tissue, other debris, etc., may remain, despite the rigorous and careful practices of dental professionals. In laser-assisted root canal treatments, a laser is used to remove dental pulp, tissue, and other debris and may be used to modify, shape, and clean the walls of the root canals. According to certain laser-based techniques, a laser produces photoacoustic shock waves in a fluid located in a root canal, and the shock waves clean the root canals. In addition, certain sonic treatment procedures generate sonic waves in a fluid located in a root canal to irrigate the root canal and remove pulp, tissue, and other debris therefrom.

There is believed to be a need to further develop dental, endodontic, and periodontic treatment methods and systems.

### SUMMARY

Example embodiments of the present invention are based on exploiting the presence of bacteria in the interior of a tooth in order to clean, disinfect, and/or deliver a payload to the interior of the tooth, e.g., as part of a dental, endodontic, and/or periodontic treatment regimen, e.g., a root canal procedure. More particularly, example embodiments of the present invention transmit ultrasonic energy to a fluid contained in the interior of the tooth, e.g., the root canal, at a frequency and amplitude that causes inertial cavitation of gas vesicles that are contained within the bacteria. The inertial cavitation of the gas vesicles may clean the interior of the tooth, e.g., the root canals, by propagating shock waves through the fluid to dislodge and/or disintegrate dental pulp, tissue, and other debris. Additionally, the inertial cavitation of the gas vesicles may disinfect the interior of the tooth, e.g., the root canals by effectively killing the bacteria present in the fluid and interior of the tooth. Moreover, the inertial cavitation of the gas vesicles may rupture the cellular membrane of the bacteria, allowing a payload, e.g., a medicament, an antibiotic, nanoparticles, etc., carried by the bacteria to disperse in the fluid and treat an ailment of the tooth.

According to an example embodiment of the present invention, a method of performing a treatment on a tooth includes: accessing an interior of the tooth through at least one opening in the tooth; delivering a fluid into the interior of the tooth through the opening in the tooth; inserting an ultrasonic instrument through the opening in the tooth; and transmitting ultrasonic energy to the fluid by the ultrasonic instrument at an amplitude and frequency adapted to cause inertial cavitation of gas vesicles located in bacteria present in the fluid.

The method may include creating the opening in the tooth.

The method may include filling the interior of the tooth with a filling material after the transmitting.

The filling material includes gutta-percha.

The method may include closing the opening in the tooth.

The opening may include a cavity in the tooth.

The interior of the tooth may include a pulp chamber and at least one root of the tooth.

The method may include removing pulp, tissue, and/or debris from the pulp chamber and/or root of the tooth before delivering the fluid into the interior of the tooth.

The bacteria may include Fusobacterium nucleatum, Prevotella intermedia, Peptostreptococcus micros, Peptostreptococcus anaerobius, Eubacterium alactolyticum, Eubacterium lentum, and/or Wolinella recta.

The method may include removing the fluid from the interior of the tooth after the transmitting of ultrasonic energy to the fluid.

The ultrasonic energy may be transmitted to the fluid at a mechanical index value less than 1.9 MPa MHz^{-1/2}.

The ultrasonic energy may be transmitted to the fluid at a mechanical index value of approximately 0.49 MPa MHz^{-1/2}.

The ultrasonic energy may be transmitted to the fluid at a frequency of approximately 670 kHz.

The treatment may include a root canal procedure.

The method may include introducing the bacteria into the interior of the tooth through the opening, the bacteria may carry a medicament, and the inertial cavitation of the gas vesicles located in the bacteria may cause rupture of cellular walls of the bacteria to deliver the medicament to the fluid and to the interior of the tooth to treat a condition of the tooth.

The medicament includes a medication.

The ultrasonic instrument may include a piezoelectric and/or capacitive transducer.

The method may be performed by a system as described herein.

According to an example embodiment of the present invention, a system for performing a treatment on a tooth includes an ultrasonic instrument adapted to be inserted into an interior of the tooth through an opening of the tooth and to generate ultrasonic energy and transmit the generated ultrasonic energy to a fluid located in the interior of the tooth at an amplitude and frequency adapted to cause inertial cavitation of gas vesicles located in bacteria present in the fluid.

The ultrasonic instrument may be adapted to generate the ultrasonic energy and transmit the generated ultrasonic energy to the fluid at a mechanical index value of approximately 0.49 MPa MHz^{-1/2} and at a frequency of approximately 670 kHz.

The system may be adapted to perform the method described herein.

Further features and aspects of example embodiments of the present invention are described in more detail below with reference to the appended schematic Figures.

### BRIEF DESCRIPTION OF THE DRAWINGS

FIG. 1 is a cross-sectional view of a tooth.

FIGS. 2A to 2C illustrate a bacterium to illustrate a method according to an example embodiment of the present invention.

FIG. 3 illustrates a system according to an example embodiment of the present invention.

### DETAILED DESCRIPTION

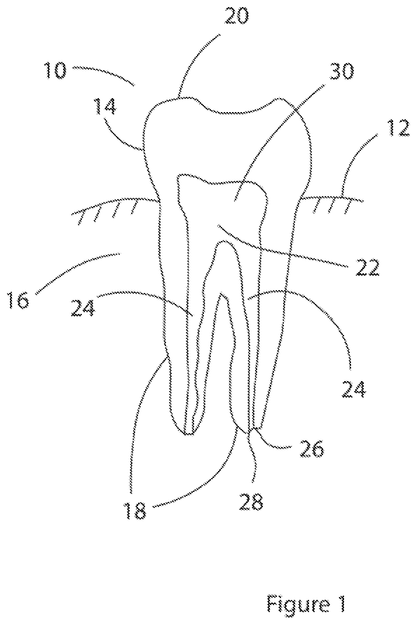

FIG. 1 is a cross-sectional view of a tooth 10, which is set in a jaw bone 16 and extends above and below a gum. 12 The tooth 10 includes a crown 14 extending above the gum 12 and one or more roots 18 extending below the gum 12 and set in the jaw bone 16. The crown 14 includes an exterior enamel layer 20. A pulp chamber 22 of tooth 10 contains dental pulp, blood vessels, and nerves that enter the tooth 10 from a hole 26 located at the apex 28 of the root 18.

A diseased, damaged, or infected tooth 10 may require intervention by a dentist, an endodontist, a periodontist, etc. For example, a root canal procedure may be required upon injury, inflammation, infection, etc., of the pulp 30. For example, pulp 30 may be damaged by decay, e.g., due to an untreated cavity, a chip in tooth 10, a crack in tooth 10, or other injury to the tooth 10. A root canal procedure includes mechanical removal of the pulp 30, e.g., by creating an opening in the crown 14 of the tooth 10, accessing the pulp 30 through a cavity in the tooth 10, etc., and removing the pulp 30 using files, such as those described, for example, in U.S. Pat. Nos. 7,967,605 and 10,052,173, each of which is expressly incorporated herein in its entirety by reference thereto. However, despite attempts at complete mechanical removal of the pulp 30, it is possible that some pulp material may remain in the pulp chamber 22, particularly in the roots 18. It is also possible that other tissue or debris remains present in the pulp chamber 22 or roots 18. Additionally, it may be necessary or appropriate to disinfect the pulp chamber, roots 18, etc., Moreover, it may be necessary or appropriate to deliver medicament to the pulp chamber 22, roots 18, etc.

Example embodiments of the present invention provide for three modes of treatment of a diseased, damaged, or infected tooth 10. In a first mode, the method and system hereof may be used to supplement the mechanical removal of pulp and other tissue from the pulp chamber 22, roots 18, etc., of tooth 10, by ablation. In a second mode, the method and system hereof may be used to disinfect the pulp chamber 22, roots 18, etc. of tooth 10. In a third mode, the method and system hereof may be used to deliver a payload to the pulp chamber 22, roots 18, etc., of tooth 10.

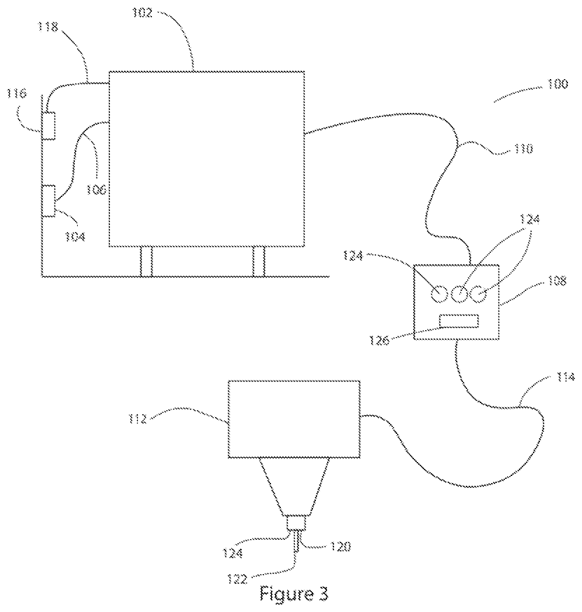

To describe these treatment modes, reference is made to FIG. 3, which illustrates a system 100 according to an example embodiment of the present invention. System 100 includes, for example, a main console 102, powered by a standard wall outlet 104 via power cord 106. The console 102 may be connected to a water source 116 via water line 118. The console 102 includes, for example, power components, logic components, hardware, software, processors, valves, water lines, etc., for performing the method described herein.

The system 100 includes a controller 108, which may be used by the operator to control operation of the system 100. The controller 108 is connected to the console 102 via line 110, which may provide both power and communication between the console 102 and controller 108. System 100 further includes an ultrasonic transducer 112 adapted to generate ultrasonic energy and deliver ultrasonic energy to tooth 100, e.g., to pulp chamber 22, roots 18, etc. The transducer 112 is connected to controller 108 via line 114, which may provide both power and communication between the controller 108 and the transducer 112. It should be appreciated that lines 110 and 114 may include fluid lines to deliver water and other fluids from console 102 to tooth 10. via a fluid outlet 120, e.g., a nozzle.

As noted above, in the first mode, the method and system hereof may be used to supplement the mechanical removal of pulp and other tissue from the pulp chamber 22, roots 18, etc., of tooth 10, by ablation. For example, after mechanical removal of pulp 30 from pulp chamber 22, roots 18, etc., some pulp, other tissue, or debris may remain in the pulp chamber 22, roots 18, etc. In order to remove this remaining material, the operator may fill the pulp chamber 22, roots 18, etc., with water or other fluid via fluid outlet 120. Bacteria present in the pulp chamber 22, roots 18, etc., including, for example, Fusobacterium nucleatum, Prevotella intermedia, Peptostreptococcus micros, Peptostreptococcus anaerobius, Eubacterium alactolyticum, Eubacterium lentum, and/or Wolinella recta, may migrate to and disperse in the water or other fluid. By targeting these bacteria with ultrasonic energy via transducer 112, the ultrasonic energy may cause cavitation of gas vesicles contained within the bacteria, thereby causing energy, pressure, or shock waves within the water or fluid. These waves may dislodge, disintegrate, etc., remaining pulp, tissue, or other debris contained within the pulp chamber 22, roots 18, etc.

In the second mode, as noted above, the method and system hereof may be used to disinfect the pulp chamber 22, roots 18, etc. of tooth 10. In order to perform disinfection, the operator may fill the pulp chamber 22, roots 18, etc., with water or other fluid via fluid outlet 120. Bacteria present in the pulp chamber 22, roots 18, etc., including, for example, those identified above, may migrate to and disperse in the water or other fluid. By targeting these bacteria with ultrasonic energy via transducer 112, the ultrasonic energy may cause cavitation of gas vesicles contained within the bacteria, thereby causing their cellular walls to rupture, effectively killing the bacteria and disinfecting the pulp chamber 22, roots 18, etc. of those bacteria.

In the third mode, as noted above, the method and system hereof the method and system hereof may be used to deliver a payload to the pulp chamber 22, roots 18, etc., of tooth 10. For example, bacteria, such as those identified above, may be introduced, e.g., from a culture, into the pulp chamber 22, roots 18, etc., of tooth 10, via fluid outlet 120. For example, a medicament, e.g., an antibiotic or other medication, nanoparticles, therapeutics, etc., may be inserted into the bacteria and carried by the bacteria with their internal structures, e.g., dissolved or dispersed in their cytoplasm. By targeting these bacteria with ultrasonic energy via transducer 112, the ultrasonic energy may cause cavitation of gas vesicles contained within the bacteria, thereby causing their cellular walls to rupture, allowing the payload to be release to the pulp chamber 22, roots 18, etc., and fluid contained therein.

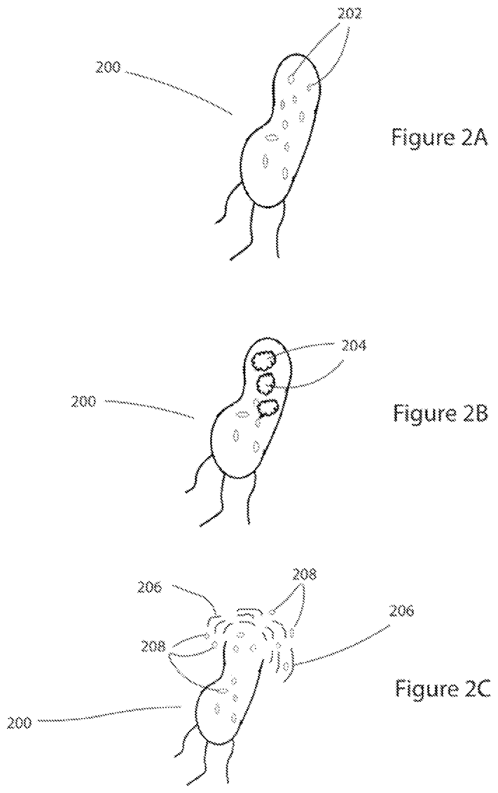

FIGS. 2A to 2C illustrate the foregoing cavitation of gas vesicles contained within bacteria, such as those identified above. Referring, for example, to FIG. 2A, a single bacterium 200 is illustrated. Contained within the cellular structure of the bacterium 200 are gas vesicles 200. These gas vesicles 202 are, for example, air-filled protein nanostructures that achieve cellular buoyancy of bacteria. Gas vesicles include amphiphilic protein shells, having a cylindrical width of, for example, 45 to 250 nm and a length of, for example, 100 to 600 nm. The shells may be permeable to gas but impermeable to liquid water, since their interior surfaces are hydrophobic.

By subjecting the gas vesicles 202 to ultrasonic energy, the gas vesicles 202 nucleate gas bubbles and cause inertial cavitation, e.g., formation, growth, and collapse of gas bubbles. For example, FIG. 2B illustrates a bacterium that is subjected to ultrasonic energy, e.g., emitted by transducer 112, causing internal cavitation of gas vesicles 202 to generate gas bubbles 204. The formation, growth, and collapse of gas bubbles 204 causes the cell membrane 206 of bacterium 200 to rupture, as illustrated, for example, in FIG. 2C.

The collapse of gas bubbles 204 may generate energy, pressure, or shock waves within the water or fluid located in the pulp chamber 22, roots 18, etc., and the energy, pressure, or shock waves propagate in the water or fluid, causing dislodgment, disintegration, etc., of remaining pulp, tissue, or other debris contained within the pulp chamber 22, roots 18, etc. Thus, the pulp chamber, roots 18, etc., may be further cleaned of pulp, tissue, or other debris contained therein, by ablation. The rupture of cell member 206 of bacterium 200 also kills the bacterium, to, for example, disinfect the pulp chamber 22, roots 18, etc., of bacteria contained therein. Moreover, bacterium 200 that is dosed or seeded with a medicament 208, e.g., antibiotics or other medications, nanoparticles, therapeutics, etc., as a payload may be introduced into the fluid contained in the pulp chamber 22, roots 18, etc., and may release that payload 208 to the fluid contained in the pulp chamber 22, roots 18, etc., upon rupture of the cell membrane 206, as illustrated in FIG. 2C. Thus, targeted delivery of medicament(s) may be achieved by utilizing bacteria 200 as the delivery mechanism or transport vehicle.

The transducer 112 may be arranged as a focused ultrasonic transducer adapted to generate ultrasonic energy in a frequency of, for example 670 kHz, and a mechanical index less than 1.9 MPa MHz^{-1/2}, which is considered to be the upper limit for diagnostic ultrasonic imaging. For example, the transducer 112 may be adapted to generate ultrasonic energy at a mechanical index of 0.49 MPa MHz^{-1/2}, which is substantially below the safety threshold mentioned above. Accordingly, the techniques described herein are considered to be effective, in ablation, disinfection, and medicament delivery, as well as safe, in that, for example, the mechanical index of 0.49 MPa MHz^{-1/2} is well below the 1.9 MPa MHz^{-1/2} safety limit mentioned above at which spontaneous cavitation in tissue may occur. Transducer 112 may be arranged as a piezoelectric transducer, a capacitive transducer, etc. In addition to fluid outlet 120, the distal end of the transducer 112 may include a tip 122 insertable into the pulp chamber 22 through a cavity or other access hole(s) created in tooth 10 by a treating dentist, endodontist, periodontist, etc., for target delivery of ultrasonic energy to pulp chamber 22, roots 18, etc. Such access hole(s) may be created in the crown 20, at its top surface, side surface, etc., the root 18, e.g., below the gum 12, etc. The hole and/or cavity may be size to allow tip 122 to be inserted therethrough and into pulp chamber 22, roots 18, etc. Prior to insertion of the tip 122 through the hole and/or cavity, the treating dentist, endodontist, periodontist, etc., may clean the pulp chamber 22, roots 18, etc., by removing pulp, tissue, other debris, etc., utilizing, for example, files, such as those described above. Once this cleaning is complete, the pulp chamber 22, roots 18, etc., may be filled with fluid, e.g., water, a solution, etc., delivered from the console 102 to the interior of tooth 10 via fluid outlet 120 directed through the hole and/or cavity. The flow of fluid into the interior of tooth 10 may be turbulent, e.g., aerated, to cause dispersal of bacteria, such as those identified above, present or remaining in the interior of tooth 10, throughout the fluid introduced into the interior of tooth 10. Thereafter, the tip 122 may be inserted through the hole or cavity, e.g., after a predetermined period of time has elapsed from introducing fluid into the interior of tooth 10, and at least partially submerged in the fluid located in the interior of the tooth 10. Ultrasonic energy may be delivered to fluid via the tip 122 at a frequency, amplitude, power, duty cycle, etc., sufficient to cause cavitation and collapse of gas vesicles contained in bacteria present in the fluid, to clean, disinfect, and/or deliver a payload to the pulp chamber 22, roots 18, etc., as described in more detail above.

After such cleaning, disinfection, and/or payload deliver, the treating dentist, endodontist, periodontist, etc., may remove the fluid remaining in the interior of tooth, using, for example, suction port 124, e.g., nozzle, located at the distal end of transducer 112. Thereafter, the treating dentist, endodontist, periodontist, etc., may continue treatment, e.g., completion of a root canal procedure, by, for example, filling the pulp chamber 22 and/or root canal(s) 24 with a filler, e.g., gutta-percha, closing the hole and/or cavity with a temporary or permanent filling and/or crown, artificial crown, etc., and any further post-procedure treatment.

The frequency, amplitude, power, duty cycle, etc., of ultrasonic energy generated by the transducer 112 may be tailored to the particular bacterium or bacteria present in the pulp chamber 22, roots 18, etc., or delivered thereto, to provide for targeted treatment of tooth 10. For example, controller 108 may include user-operable controls 124, arranged, e.g., as knobs, sliders, switches, etc., for setting or adjusting operational parameters of the system 100, such as, frequency, amplitude, power, duty cycle, water delivery, suction, etc. The controller 108 may also include a display 126 adapted to display information relating to the system 100, including, for example, operational parameters thereof. The controls 124 and display 126 may be integrated as a graphical user interface (GUI).

While it is described above that tip 122 is inserted into the interior of the tooth 10 and submerged in the fluid while delivering ultrasonic energy to the fluid, it should be appreciated that ultrasonic energy may be imparted to the fluid from a location exterior, e.g., from the top, along the side, of the tooth 10, such that ultrasonic energy is transmitted through the structures of the tooth 10.

1. A method of performing a treatment on a tooth, comprising:
accessing an interior of the tooth through at least one opening in the tooth;
delivering a fluid into the interior of the tooth through the opening in the tooth;
inserting an ultrasonic instrument through the opening in the tooth; and
transmitting ultrasonic energy to the fluid by the ultrasonic instrument at an amplitude and frequency adapted to cause inertial cavitation of gas vesicles located in bacteria present in the fluid.

2. The method according to claim 1, further comprising creating the opening in the tooth.

3. The method according to claim 1, further comprising filling the interior of the tooth with a filling material after the transmitting.

4. The method according to claim 3, wherein the filling material includes gutta-percha.

5. The method according to claim 1, further comprising closing the opening in the tooth.

6. The method according to claim 1, wherein the opening includes a cavity in the tooth.

7. The method according to claim 1, wherein the interior of the tooth includes a pulp chamber and at least one root of the tooth.

8. The method according to claim 7, further comprising removing pulp, tissue, and/or debris from the pulp chamber and/or root of the tooth before delivering the fluid into the interior of the tooth.

9. The method according to claim 1, wherein the bacteria include Fusobacterium nucleatum, Prevotella intermedia, Peptostreptococcus micros, Peptostreptococcus anaerobius, Eubacterium alactolyticum, Eubacterium lentum, and/or Wolinella recta.

10. The method according to claim 1, further comprising removing the fluid from the interior of the tooth after the transmitting of ultrasonic energy to the fluid.

11. The method according to claim 1, wherein the ultrasonic energy is transmitted to the fluid at a mechanical index value less than 1.9 MPa MHz ½.

12. The method according to claim 1, wherein the ultrasonic energy is transmitted to the fluid at a mechanical index value of approximately 0.49 MPa MHz ½.

13. The method according to claim 1, wherein the ultrasonic energy is transmitted to the fluid at a frequency of approximately 670 kHz.

14. The method according to claim 1, wherein the treatment includes a root canal procedure.

15. The method according to claim 1, further comprising introducing the bacteria into the interior of the tooth through the opening, the bacteria carrying a medicament, the inertial cavitation of the gas vesicles located in the bacteria causing rupture of cellular walls of the bacteria to deliver the medicament to the fluid and to the interior of the tooth to treat a condition of the tooth.

16. The method according to claim 15, wherein the medicament includes a medication.

17. The method according to claim 1, wherein the ultrasonic instrument includes a piezoelectric and/or capacitive transducer.

18. A system for performing a treatment on a tooth, comprising:
an ultrasonic instrument adapted to be inserted into an interior of the tooth through an opening of the tooth and to generate ultrasonic energy and transmit the generated ultrasonic energy to a fluid located in the interior of the tooth at an amplitude and frequency adapted to cause inertial cavitation of gas vesicles located in bacteria present in the fluid.

19. The system according to claim 18, wherein the ultrasonic instrument is adapted to generate the ultrasonic energy and transmit the generated ultrasonic energy to the fluid at a mechanical index value of approximately 0.49 MPa MHz ½ and at a frequency of approximately 670 kHz.

20. The system according to claim 18, wherein the system is adapted to perform a method including:
accessing the interior of the tooth through the opening of the tooth;
delivering the fluid into the interior of the tooth through the opening of the tooth;
inserting the ultrasonic instrument through the opening of the tooth; and
transmitting the ultrasonic energy to the fluid by the ultrasonic instrument at the amplitude and frequency adapted to cause the inertial cavitation of the gas vesicles located in the bacteria present in the fluid.

21. The system according to claim 18, wherein the ultrasonic instrument includes a piezoelectric and/or capacitive transducer.

22. The method according to claim 1, wherein the method is performed by a system that includes the ultrasonic instrument, the ultrasonic instrument being adapted to be inserted into the interior of the tooth through the opening of the tooth and to generate ultrasonic energy and transmit the generated ultrasonic energy to the fluid located in the interior of the tooth at the amplitude and the frequency adapted to cause the inertial cavitation of the gas vesicles located in the bacteria present in the fluid.

23. A system for performing a treatment on the tooth, comprising:
an ultrasonic instrument adapted to be inserted into an interior of the tooth through an opening of the tooth and to generate ultrasonic energy and transmit the generated ultrasonic energy to a fluid located in the interior of the tooth at an amplitude and frequency adapted to cause inertial cavitation of gas vesicles located in bacteria present in the fluid;
wherein the system is adapted to perform a method including:
accessing an interior of the tooth through at least one opening in the tooth;
delivering a fluid into the interior of the tooth through the opening in the tooth;
inserting an ultrasonic instrument through the opening in the tooth; and
transmitting ultrasonic energy to the fluid by the ultrasonic instrument at an amplitude and frequency adapted to cause inertial cavitation of gas vesicles located in bacteria present in the fluid.

24. A method of performing a treatment on a tooth, comprising:
accessing an interior of the tooth through at least one opening in the tooth;
delivering a fluid into the interior of the tooth through the opening in the tooth;
inserting an ultrasonic instrument through the opening in the tooth; and
transmitting ultrasonic energy to the fluid by the ultrasonic instrument at an amplitude and frequency adapted to cause inertial cavitation of gas vesicles located in bacteria present in the fluid;
wherein the method is performed by a system that includes the ultrasonic instrument, the ultrasonic instrument being adapted to be inserted into the interior of the tooth through the opening of the tooth and to generate ultrasonic energy and transmit the generated ultrasonic energy to the fluid located in the interior of the tooth at the amplitude and the frequency adapted to cause the inertial cavitation of the gas vesicles located in the bacteria present in the fluid.
